(19) United States
(12) Patent Application Publication
(10) Pub. No.: US 2011/0290003 A1
(43) Pub. Date: Dec. 1, 2011

(54) GAS SENSOR WITH A ZINC-OXIDE NANOSTRUCTURE AND METHOD FOR PRODUCING THE SAME

(75) Inventors: Wen-Chau Liu, Tainan City (TW); Huey-Ing Chen, Tainan City (TW); Tai-You Chen, Tainan City (TW); Tsung-Han Tsai, Tainan City (TW); I-Ping Liu, Tainan City (TW); Chi-Hsiang Hsu, Tainan City (TW)

(73) Assignee: NATIONAL CHENG KUNG UNIVERSITY, Tainan City (TW)

(21) Appl. No.: 13/115,696

(22) Filed: May 25, 2011

(30) Foreign Application Priority Data
May 26, 2010 (TW) 099116786

(51) Int. Cl. G01N 33/00; B05D 5/12; B82Y 30/00

(52) U.S. Cl. 73/31.06; 427/126.3; 977/762; 977/773

(57) ABSTRACT

A gas sensor includes a substrate; a seed layer positioned on the substrate; a zinc-oxide nanostructure formed on the seed layer; a metal nanoparticle formed on the zinc-oxide nanostructure; a first electrode positioned on the zinc-oxide nanostructure; and a second electrode positioned on the zinc-oxide nanostructure apart from the first electrode to electrically connect to the first electrode.

### CROSS-REFERENCE TO RELATED APPLICATION

This non-provisional application claims priority under 35 U.S.C. §119(a) on Patent Application No. 099116786 filed in Taiwan R.O.C. on May 26, 2010, the entire contents of which are hereby incorporated by reference.

### FIELD OF THE INVENTION

The invention relates to a gas sensor, more particularly, to a gas sensor with a zinc-oxide nanostructure.

### BACKGROUND OF THE INVENTION

Harmful gas, such as ammonia and carbon monoxide, exists in the atmosphere and is colorless and odorless that people are difficult to detect the very existence of such gas. If the concentration of the harmful gas reaches to a certain level, people living in such an environment may suffer headache, dizziness, nausea, spasm and spewing, more seriously, resulting in shock and decease.

Furthermore, when the concentration of flammable or explosive gas, such as ethylene and hydrogen, produced in a chemical factory or a laboratory reaches to a dangerous level and there's a negligence in management of fire or the like, not only the construction site but also the people around this kind of gas will definitely be seriously hurt. As such, a gas sensor is necessary for sensing the concentration of a harmful gas. The gas sensor can sense the concentration of the harmful gas in real time and send out signals to warn people of the existence of such dangerous gas so as to prevent any potential danger.

A conventional gas sensor is a metal-oxide semiconductor gas sensor, and the metal-oxide semiconductor gas sensor senses gas by a metal-oxide semiconductor powder therein, such as tin (IV) oxide powder and zinc oxide powder. In such a way, when the metal-oxide semiconductor gas sensor is at a working temperature (e.g. 200° C. to 400° C.), the metal-oxide semiconductor powder can absorb the sensed gas, resulting in a voltage change of the metal-oxide semiconductor gas sensor and a determination of the concentration of the sensed gas. However, the metal-oxide semiconductor powder occupies a large volume and has a low surface area, which leads to a low contact area for the sensed gas so that the metal-oxide semiconductor gas sensor has a low gas sensitivity. In addition, the metal-oxide semiconductor powder also has a low identification for the sensed gas and other gas, and consequently the final result by the sensor is uncertain.

### SUMMARY OF THE INVENTION

An objective of the invention is to provide a gas sensor, which has a high gas sensitivity.

A further objective of the invention is to provide a gas sensor, which has a high gas identification capability for various gases.

The gas sensor of the invention comprises:

a substrate;

a seed layer positioned on the substrate;

a zinc-oxide nanostructure formed on the seed layer;

a metal nanoparticle formed on the zinc-oxide nanostructure;

a first electrode positioned on the zinc-oxide nanostructure; and

a second electrode positioned on the zinc-oxide nanostructure apart from the first electrode to electrically connect to the first electrode.

With the scope of the invention is a method for producing a gas sensor, and the method comprises:

providing a substrate;

forming a seed layer on the substrate;

forming a zinc-oxide nanostructure on the seed layer;

forming a metal nanoparticle on the zinc-oxide nanostructure;

forming a first electrode on the zinc-oxide nanostructure; and

forming a second electrode on the zinc-oxide nanostructure apart from the first electrode to electrically connect to the first electrode.

### BRIEF DESCRIPTION OF THE DRAWINGS

FIG. 1 is a perspective view of a gas sensor of the invention.

FIG. 2 is a partially perspective view of the gas sensor shown in FIG. 1.

FIG. 3 is a perspective view of the zinc-oxide nanostructure and the metal nanoparticle of the gas sensor shown in FIG. 1.

FIG. 4 is a chart showing an ammonia sensitivity of a gas sensor at different working temperature.

### DETAILED DESCRIPTION OF THE INVENTION

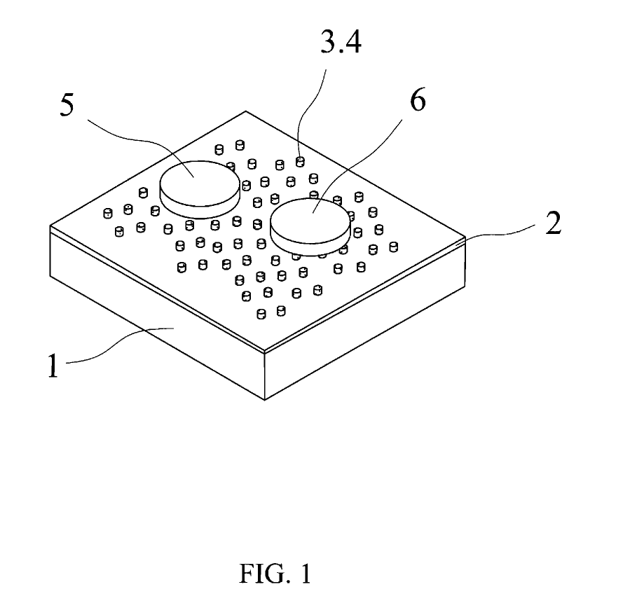

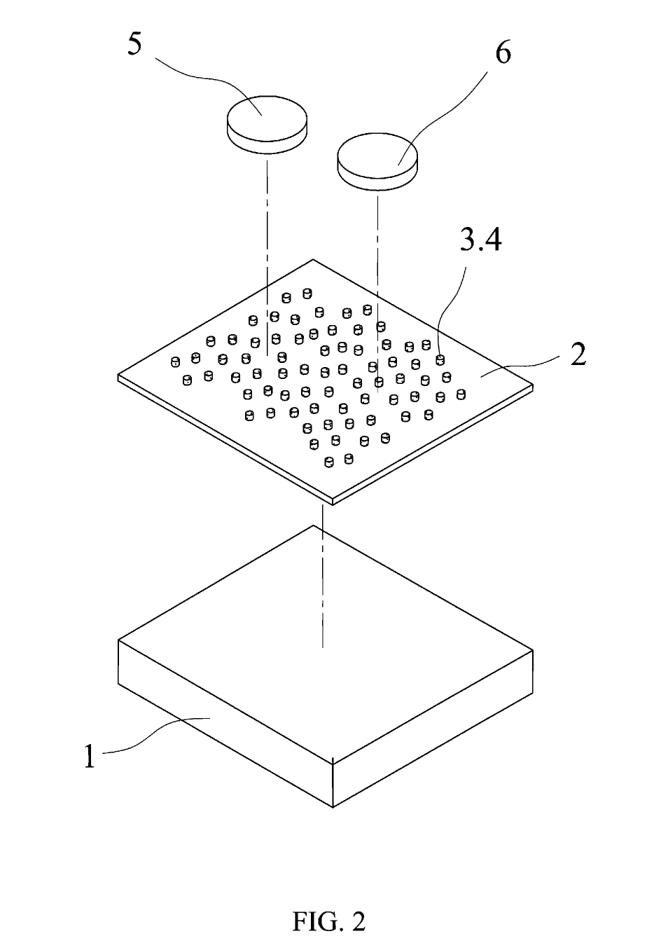

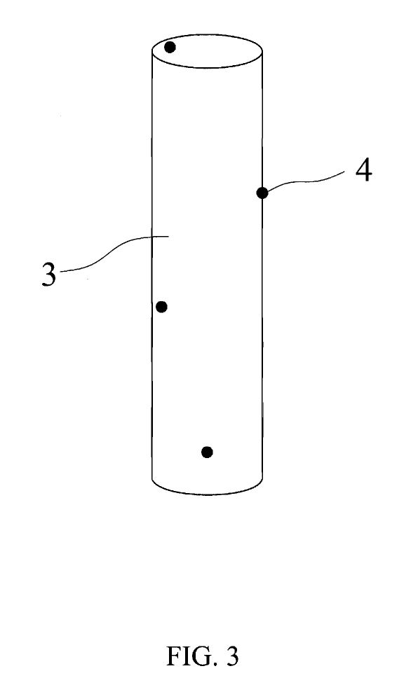

As shown in FIGS. 1 to 3, a gas sensor comprises:

a substrate (1);

a seed layer (2) positioned on the substrate (1);

a zinc-oxide nanostructure (3) formed on the seed layer (2);

a metal nanoparticle (4) formed on the zinc-oxide nanostructure (3);

a first electrode (5) positioned on the zinc-oxide nanostructure (3); and

a second electrode (6) positioned on the zinc-oxide nanostructure (3) apart from the first electrode (4) to electrically connect to the first electrode (4).

In some embodiments of the invention, the substrate (1) is an insulator. Preferably, the substrate (1) is selected from a group consisting of a sapphire, a silicon wafer, a glass and a IIIA-VA semiconductor.

In some embodiments of the invention, the seed layer (2) has a thickness ranging from 1 nm to 500 μm.

In some embodiments of the invention, the seed layer (2) is zinc oxide or IIIA metal-doped zinc oxide. Preferably, the IIIA metal-doped zinc oxide is selected from a group consisting of aluminum-doped zinc oxide, gallium-doped zinc oxide and indium-doped zinc oxide.

In some embodiments of the invention, the zinc-oxide nanostructure (3) is in a shape of a nanowire, a nanorod, a nanoparticle or a nanotube. Preferably, the nanorod has a length ranging from 100 nm to 1 μm and a diameter ranging from 10 nm to 100 nm.

In some embodiments of the invention, the metal nanoparticle (4) has a diameter ranging from 2 nm to 5 nm.

In some embodiments of the invention, the metal nanoparticle (4) is selected from a group consisting of palladium, platinum, gold, rhodium, silver and iridium.

In some embodiments of the invention, the first electrode (5) is selected from a group consisting of aluminum, platinum, chromium, nickel, gold, titanium, palladium and copper.

In some embodiments of the invention, the second electrode (6) is selected from a group consisting of aluminum, platinum, chromium, nickel, gold, titanium, palladium and copper.

As further sown in FIGS. 1 to 3, a method for producing a gas sensor comprises:

providing a substrate (1);

forming a seed layer (2) on the substrate (1);

forming a zinc-oxide nanostructure (3) on the seed layer (2);

forming a metal nanoparticle (4) on the zinc-oxide nanostructure (3);

forming a first electrode (5) on the zinc-oxide nanostructure (3); and

forming a second electrode (6) on the zinc-oxide nanostructure (3) apart from the first electrode (5) to electrically connect to the first electrode (5).

In some embodiments of the invention, the substrate (1) is an insulator. Preferably, the substrate (1) is selected from a group consisting of a sapphire, a silicon wafer, a glass and a IIIA-VA semiconductor.

In some embodiments of the invention, the seed layer (2) is formed on the substrate (1) by a sputtering method or a coating method.

In some embodiments of the invention, the seed layer (2) has a thickness ranging from 1 nm to 500 μm.

In some embodiments of the invention, the seed layer (2) is zinc oxide or IIIA metal-doped zinc oxide. Preferably, the IIIA metal-doped zinc oxide is selected from a group consisting of aluminum-doped zinc oxide, gallium-doped zinc oxide and indium-doped zinc oxide.

In some embodiments of the invention, the zinc-oxide nanostructure (3) is formed on the seed layer (2) by a hydrothermal method, a metal-organic chemical vapor deposition method, a chemical vapor deposition method, a pulsed laser deposition method, a molecular beam epitaxy method or an electrochemical method.

It is noticed that the hydrothermal method comprises: providing a growth solution composed of a zinc salt solution and an alkaline solution; dipping the seed layer (2) into the growth solution; and heating the growth solution to form the zinc-oxide nanostructure (3) on the seed layer (2).

Preferably, the zinc salt solution is a zinc nitrate hexahydrate (Zn(NO₃)₂.6H₂O) solution or a zinc acetate dihydrate (Zn(CH₃COO)₂.2H₂O) solution.

Preferably, the alkaline solution is a sodium hydroxide solution or a hexamethylenetetramine solution.

Preferably, the growth solution is heated at 60° C. to 150° C. for 1 hour to 24 hours.

In some embodiments of the invention, the zinc-oxide nanostructure (3) has a shape of a nanowire, a nanorod, a nanoparticle or a nanotube. Preferably, the nanorod has a length ranging from 100 nm to 1 μm and a diameter ranging from 10 nm to 100 nm.

In some embodiments of the invention, the metal nanoparticle (4) is formed on the zinc-oxide nanostructure (3) by an impregnation method.

It is noticed that the impregnation method comprises: providing a precursor solution composed of a precursor; coating the precursor solution on the zinc-oxide nanostructure (3); and heating the precursor solution and applying a reaction gas to the precursor solution to perform a reduction reaction to form the metal nanoparticle (4) on the zinc-oxide nanostructure (3).

Preferably, the precursor solution is heated at 50° C. to 1000° C.

Preferably, the precursor is chloroplatinic acid.

Preferably, the reaction gas is hydrogen.

In some embodiments of the invention, the metal nanoparticle (4) has a diameter ranging from 2 nm to 5 nm.

In some embodiments of the invention, the metal nanoparticle (4) is selected from a group consisting of palladium, platinum, gold, rhodium, silver and iridium.

In some embodiments of the invention, the first electrode (5) is selected from a group consisting of aluminum, platinum, chromium, nickel, gold, titanium, palladium and copper.

In some embodiments of the invention, the second electrode (6) is selected from a group consisting of aluminum, platinum, chromium, nickel, gold, titanium, palladium and copper.

An example for further illustration of the invention is below but not intended to limit the invention. Any modifications and applications by persons skilled in the art of the invention should be within the scope of the invention.

Firstly, a sapphire is provided as the substrate of the example, and a zinc oxide, as the seed layer of the example, is sputtered on the sapphire.

Thereafter, the sputtered zinc oxide is dipped into a growth solution composed of a zinc nitrate hexahydrate solution and a zinc acetate dihydrate solution and heated to 70° C. for 6 hours as a result that a zinc-oxide nanorod with a length of 1 μm and a diameter of 60 nm is formed on the sputtered zinc oxide.

After coating a chloroplatinic acid solution on the zinc-oxide nanorod, the coated chloroplatinic acid solution is heated at 300° C. and hydrogen is applied to the coated chloroplatinic acid solution. In such a way, the coated chloroplatinic acid solution is reduced to a platinum nanoparticle on the zinc-oxide nanorod.

Finally, two pieces of aluminum are formed on the zinc-oxide nanorod by an evaporation method, and thus the gas sensor of the example is produced.

In order to determine the gas sensitivity of the gas sensor of the example, the gas sensor is placed in a 1,000 ppm ammonia/air atmosphere.

Sensing the ammonia by the gas sensor is detailedly described below.

The surface of the zinc-oxide nanorod absorbs oxygen in the air at a working temperature. The absorbed oxygen captures an electron from the zinc-oxide nanorod and is charged as an oxygen ion (i.e., O⁻, O₂⁻, and O²⁻). Thus, a resistance named as Rₐᵢᵣ is obtained.

When the ammonia is exposed to the gas sensor, the ammonia is absorbed by the platinum nanoparticle and conducted to the zinc-oxide nanorod via spillover effect. The electron in the oxygen ion is released to the zinc nanorod, and thus another resistance named as Rₐₘₘₒₙᵢₐ lower than that of the foregoing Rₐᵢᵣ is obtained. Herein, an ammonia sensitivity (denoted as “S”) of the gas sensor is defined as the ratio of the foregoing Rₐᵢᵣ to the foregoing Rₐₘₘₒₙᵢₐ and described as the following formula:

$$S = \frac{{R}_{air}}{{R}_{ammonia }} \times 100 \%$$

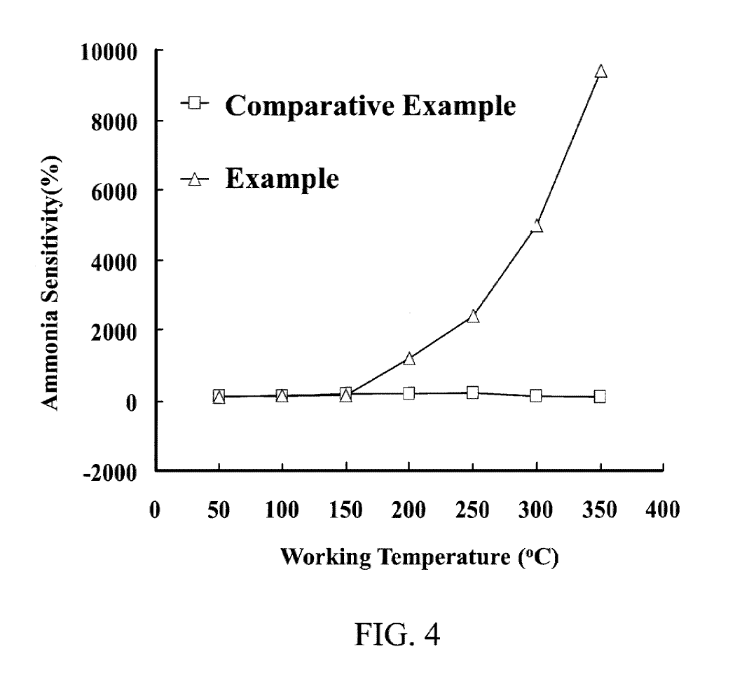

With reference to FIG. 4, the ammonia sensitivity of the gas sensor of the example at different working temperature is presented, wherein a gas sensor with the same manner except for no metal nanoparticle thereon is also provided as a comparative example. It is learned that the ammonia sensitivity of the example is 1210% at 200° C. and increases as the working temperature rises; however, the ammonia sensitivity of the comparative example is only 214% at 250° C. and decreases as the working temperature rises.

What is claimed is:

1. A gas sensor, comprising:
a substrate;
a seed layer positioned on the substrate;
a zinc-oxide nanostructure formed on the seed layer;
a metal nanoparticle formed on the zinc-oxide nanostructure;
a first electrode positioned on the zinc-oxide nanostructure; and
a second electrode positioned on the zinc-oxide nanostructure apart from the first electrode to electrically connect to the first electrode.

2. The gas sensor as claimed as claim 1, wherein the zinc-oxide nanostructure is in a shape of a nanowire, a nanorod, a nanoparticle or a nanotube.

3. The gas sensor as claimed as claim 1, wherein the seed layer is zinc oxide or IIIA metal-doped zinc oxide.

4. The gas sensor as claimed as claim 3, wherein the IIIA metal-doped zinc oxide is selected from a group consisting of aluminum-doped zinc oxide, gallium-doped zinc oxide and indium-doped zinc oxide.

5. The gas sensor as claimed as claim 1, wherein the metal nanoparticle is selected from a group consisting of palladium, platinum, gold, rhodium, silver and iridium.

6. The gas sensor as claimed as claim 2, wherein the nanorod has a length ranging from 100 nm to 1 μm and a diameter ranging from 10 nm to 100 nm.

7. The gas sensor as claimed as claim 1, wherein the metal nanoparticle has a diameter ranging from 2 nm to 5 nm.

8. A method for producing a gas sensor, comprising:
providing a substrate;
forming a seed layer on the substrate;
forming a zinc-oxide nanostructure on the seed layer;
forming a metal nanoparticle on the zinc-oxide nanostructure;
forming a first electrode on the zinc-oxide nanostructure; and
forming a second electrode on the zinc-oxide nanostructure apart from the first electrode to electrically connect to the first electrode.

9. The method as claimed as claim 8, wherein the zinc-oxide nanostructure forming step is by a hydrothermal method, a metal-organic chemical vapor deposition method, a chemical vapor deposition method, a pulsed laser deposition method, a molecular beam epitaxy method or an electrochemical method.

10. The method as claimed as claim 8, wherein the metal nanoparticle forming step is by an impregnation method.

11. The method as claimed as claim 9, wherein the hydrothermal method comprises:
providing a growth solution composed of a zinc salt solution and an alkaline solution;
dipping the seed layer into the growth solution; and
heating the growth solution to form the zinc-oxide nanostructure on the seed layer.

12. The method as claimed as claim 11, wherein the zinc salt solution is a zinc nitrate hexahydrate (Zn(NO₃)₂.6H₂O) solution or a zinc acetate dihydrate (Zn(CH₃COO)₂.2H₂O) solution.

13. The method as claimed as claim 11, wherein the alkaline solution is a sodium hydroxide solution or a hexamethylenetetramine solution.

14. The method as claimed as claim 11, wherein the growth solution heating step is at 60° C. to 150° C. for 1 hour to 24 hours.

15. The method as claimed as claim 10, wherein the impregnation method comprises:
providing a precursor solution composed of a precursor;
coating the precursor solution on the zinc-oxide nanostructure; and
heating the precursor solution and applying a reaction gas to perform a reduction reaction to form the metal nanoparticle on the zinc-oxide nanostructure.

16. The method as claimed as claim 15, wherein the precursor is chloroplatinic acid.

17. The method as claimed as claim 15, wherein the precursor solution heating step is at 50° C. to 1000° C.

18. The method as claimed as claim 15, wherein the reaction gas is hydrogen.

19. The method as claimed as claim 8, wherein the seed layer forming step is by a sputtering method or a coating method.
